(19) United States
(12) Patent Application Publication
(10) Pub. No.: US 2004/0254478 A1
(43) Pub. Date: Dec. 16, 2004

(54) Fluorescence filter for tissue examination and imaging

(76) Inventors: Elbert de Josselin de Jong, Bussum (NL); Monique van der Veen, Almere (NL); Elbert Waller, Amsterdam (NL)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/851,850

(22) Filed: May 21, 2004

Related U.S. Application Data
(60) Provisional application No. 60/472,486, filed on May 22, 2003.

(51) Int. Cl. A61B 6/00

(52) U.S. Cl. 600/476; 359/361; 433/29

(57) ABSTRACT

A tissue examination system is disclosed in various embodiments, generally including a handle, a lighting element, (such as an LED), a filter, and in some embodiments a mirror. In a preferred embodiment, a lighting element and filter are attached to the distal end of a handle so that fluorescence by the bodily tissue can be directly observed by the filter in various configurations. In another, a mirror is integrated with the filter and light assembly to reflect and filter fluoresced light to an observer, while in yet another, a shield blocks ambient light from interfering with the tissue examination by injecting additional light into the view.

### REFERENCE TO RELATED APPLICATION

This application contains subject matter related to U.S. patent application Ser. No. 10/209,574, filed Jul. 31, 2002, (the “Inspection” application) which is hereby incorporated by reference in its entirety.

### FIELD OF THE INVENTION

The present invention relates generally to devices and methods for inspecting bodily (especially dental and dermal) tissue and in particular to such devices and methods involving the detection of differential fluorescence emissions from abnormal and healthy tissues.

### BACKGROUND

Various devices and methods have been proposed that use fluorescence emissions for to detect e.g. caries, plaque, calculus and also other abnormal conditions of bodily tissue such as cancer. In such devices the amount of light at predetermined wavelength ranges illuminating the tissues needs to be controlled, such that the amount of induced fluorescence controllably depends on the tissue properties. Furthermore for optimum contrast between illumination light and remitted fluorescence the amount of light at the illumination wavelength ranges needs to be blocked from reaching the detector and/or observer. Nevertheless most proposed and existing devices control these aspects only in part, if at all.

For example, U.S. Pat. No. 3,711,700 relates to a disclosing light for inspecting tooth surfaces. The disclosing light is designed for use in conjunction with a fluorescent dye such as sodium fluorescein. The disclosing light includes a light source and a dichroic reflector behind the light source to reflect blue light forward and the remainder backward. A second dichroic filter is positioned in front of the light source and transmits blue light and reflects back infrared radiation. A mirror reflects yellow light to the observer and transmits other wavelengths. This device does not take any measures to control the amount of ambient light entering the oral cavity and thus interfering with the fluorescence.

U.S. Pat. No. 4,266,535 discloses a diagnostic lamp for use in conjunction with a fluorescent dye to inspect tooth surfaces. The lamp includes an incandescent bulb light source used in conjunction with a lens to supply a divergent beam of rays. A blue filter with an applied dichroic filter is used to filter the rays, which are made to impinge upon the fluorescent dye. The resulting fluorescence can be viewed in a folding, adjustable mirror. Also this device does not control the amount of ambient light entering the oral cavity, and the induced fluorescent together with the reflected excitation light are observed as a whole rather than the induced fluorescence alone.

U.S. Pat. No. 5,957,687 discloses another diagnostic unit including a filtered light source and a mirror contained in a housing. Filtered light is concentrated in the blue frequency range and causes fluorescent dye to fluoresce. This can then be viewed in the mirror.

U.S. Pat. No. 4,290,433 discloses a method and device for detecting caries in human teeth using luminescence. The luminescence from teeth is measured at two predetermined wavelengths, one of which yields luminescence that is about the same for caries-containing and non-caries-containing surfaces, while the other yields luminescence that is higher for caries-containing surfaces. A signal is generated corresponding to the difference in the intensities at the two measured wavelengths. A baseline measurement is taken at a non-decayed region, and the probe is then directed to other regions to assess the presence or absence of caries.

U.S. Pat. No. 4,515,476 describes a device for the ocular determination of discrepancies in the luminescence capacity of tooth surfaces. The disclosed device includes an argon laser for generating visible light in a predetermined wavelength range that causes luminescence from tooth surfaces. An absorption filter is used to eliminate reflections and luminescence within the predetermined wavelength range of the laser light. In this fashion, unsound tooth surface areas such as caries can be viewed as dark areas.

U.S. Pat. No. 5,894,620 discloses an electric toothbrush with means for locating dental plaque. The electric toothbrush includes an optical fiber for transmitting excitation radiation to tooth surfaces, and an optical fiber(s) for receiving a return luminescence or fluorescence signal. The return signal is then processed to activate a light or audible signal indicative of the presence of plaque.

Shields have been used in combinations with illumination devices and the most common shields know are sunshades and sunglasses preventing a detector (e.g. a photographic camera) or the human eye from intense sunlight. In the dental field shields are commonly used with curing light systems such as the shields that are described in U.S. Pat. Nos. 5,288,231; 6,155,823 and 4,615,679. Each of these patents describe a light shield for use with a curing light device, where the shield substantially attenuates light (in the blue range) thereby protecting the operator from harmful and distracting direct and indirect light-radiation from the curing light. Apart from differences in shape the curing lights shields all have in common that they protect the eyes of the user from harmful light-radiation generated by the curing light gun. These shields do not control the amount of ambient light or curing light reaching the restorative materials in the tooth tissue, and are not designed to substantially transmit fluorescence light induced in the tooth tissues for observational purposes.

In light of this background, a need remains for a shield that can be combined with a variety of detection techniques to allow only a controlled amount of excitation light of a specific wavelength range to expose the tissue to be examined, reducing the exposure of such tissue to ambient light while remitted fluorescence from the tissue under examination is substantially transmitted towards the observer or a detection device incorporated in the illumination device. The present invention addresses those needs.

### SUMMARY

Accordingly, in one embodiment, the invention provides a shield used with a handheld implement for use in detecting abnormal tissue conditions. The implement contains a light source producing light of a specific wavelength range, effective to cause visually detectable differential fluorescence emissions from normal and abnormal bodily tissues. The shield allows the tissue under examination to be exposed to light from the light source, while ambient light is attenuated. The remitted fluorescence from the tissue is transmitted through the walls of the shield, yet light of the excitation wavelengths is blocked to improve direct visualization of the differential fluorescence of normal and abnormal bodily tissue.

In another embodiment, the invention provides a shield for use with an intra-oral fluorescence camera with integrated illumination device. In this embodiment the shield blocks ambient light, while the light from the illumination device is guided towards the tissue to be examined. Remitted fluorescence from the tissue is guided towards the camera sensor. For quantification purposes the shield in this embodiment also functions as a distance holder to ensure a fixed distance between the light source and the tissue on one hand and the tissue and the camera sensor on the other hand, thus ensuring a controlled amount of radiant power illuminating the tissue and corresponding controlled amount of remitted fluorescence depending on the tissue properties.

In some forms of this embodiment, the shield is made of a deformable material. In others, it takes the shape of a cylinder, cone, or partial sphere. In some embodiments a mirror is placed on or within one or more internal surfaces to provide alternative viewing angles in some embodiments. In other embodiments, at least a portion of the shield has magnifying properties, so that the oral structure being observed looks larger to the observer, while in other embodiments, the mirror has magnifying properties.

The present invention provides improved and alternative implements, apparatuses and methods for inspecting tissues for abnormalities, such as inspecting tooth surfaces for caries or bacterial metabolites potentially indicative of plaque. Additional embodiments as well as features and advantages of the invention will be apparent to those of ordinary skill in the art from the descriptions herein.

### BRIEF DESCRIPTION OF THE DRAWINGS

FIG. 1 is a perspective view of an implement according to a first embodiment of the present invention.

FIG. 2 is a side view of an implement being used to examine a tooth according to the first embodiment of the present invention.

FIG. 3 is a perspective view of an implement according to a second embodiment of the present invention.

FIG. 4 is a side view of an implement being used to examine a tooth according to the second embodiment of the present invention.

FIG. 5 is a perspective view of an implement according to a third embodiment of the present invention.

FIG. 6 is a side view of an implement being used to examine a tooth according to the third embodiment of the present invention.

FIG. 7 is a side view of an implement being used to examine a tooth according to a fourth embodiment of the present invention.

FIG. 8 is a perspective view of an implement according to a fifth embodiment of the present invention, being used to examine a tooth.

FIG. 9 is a side view of the fifth embodiment of the present invention.

FIG. 10 is a side view of an implement according to a sixth embodiment of the present invention, with a magnified view of a layered optical component according to the sixth embodiment of the present invention.

FIG. 11 is a side view of a layered optical component according to a seventh embodiment of the present invention.

FIG. 12 is a side view of a layered optical component according to an eighth embodiment of the present invention.

FIG. 13 is a side view of a dermatological application according to a ninth embodiment of the present invention.

FIG. 14 is a side, cutaway view of a hood/filter according to a tenth embodiment of the present invention.

FIG. 15 a perspective view of a hood/filter according to an eleventh embodiment of the present invention.

### DESCRIPTION

For the purpose of promoting an understanding of the principles of the present invention, reference will now be made to the embodiment illustrated in the drawings and specific language will be used to describe the same. It will, nevertheless, be understood that no limitation of the scope of the invention is thereby intended; any alterations and further modifications of the described or illustrated embodiments and any further applications of the principles of the invention as illustrated therein are contemplated as would normally occur to one skilled in the art to which the invention relates.

The present invention generally relates to an implement for inspection of bodily tissue, and more particularly to an implement and method for tissue inspection using a filter structure that can efficiently shield light emitted by an LED from direct visualization, and provide a more clear view of the tissue. Since some of the features of the invention are similar to those described in the Inspection application, a detailed description of certain common elements is omitted from these specifications, though incorporated herein by reference above.

It is noted that, in this document, “filter” means to allow only selected wavelengths of light to pass through an object or layer.

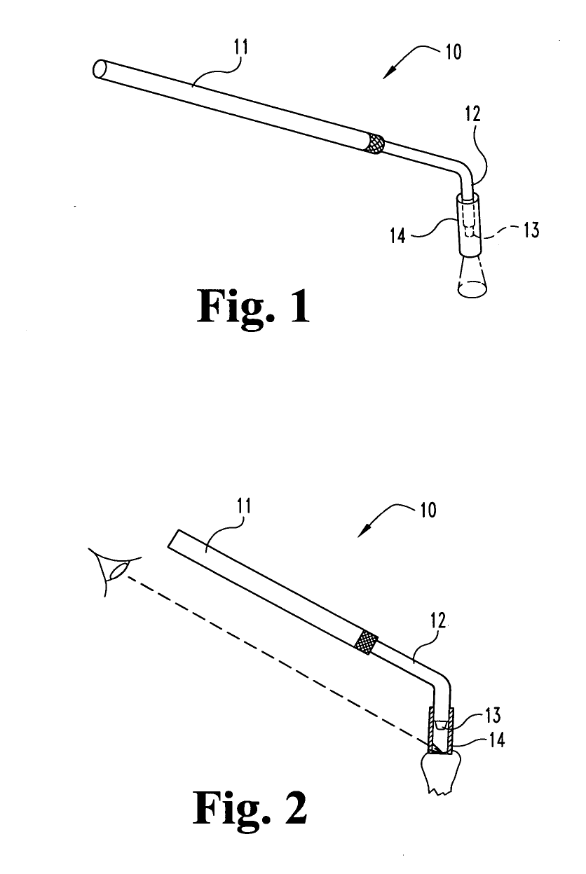

FIGS. 1 and 2 show a first embodiment of the present invention, a dental implement. In FIG. 1, the dental implement 10 includes a handle portion 11, and tip portion 12 connected at a angle to the handle portion 11. Light-emitting diode (LED) 13 is affixed to the tip of portion 12, and filter structure 14 is attached to tip portion 12 adjacent to LED 13, surrounding the LED 13. LED 13 preferably emits light having a peak intensity between about 380 and 500 nm, and more preferably between 390 and 450 nm.

When the implement is used, at least part of tip portion 12 is inserted into the mouth with an open end of the filter structure 14 in direct contact with the tooth surface as shown in FIG. 2. Implement 10 emits light from LED 13 onto the tooth surface, and the autofluorescence generated by the tooth surface is observed through the filter structure 14. Since the open end of filter structure 14 directly contacts the tooth surface, and the light from LED 13 does not leak outside the filter structure 14, direct visualization of the light from the LED 13 is efficiently prevented.

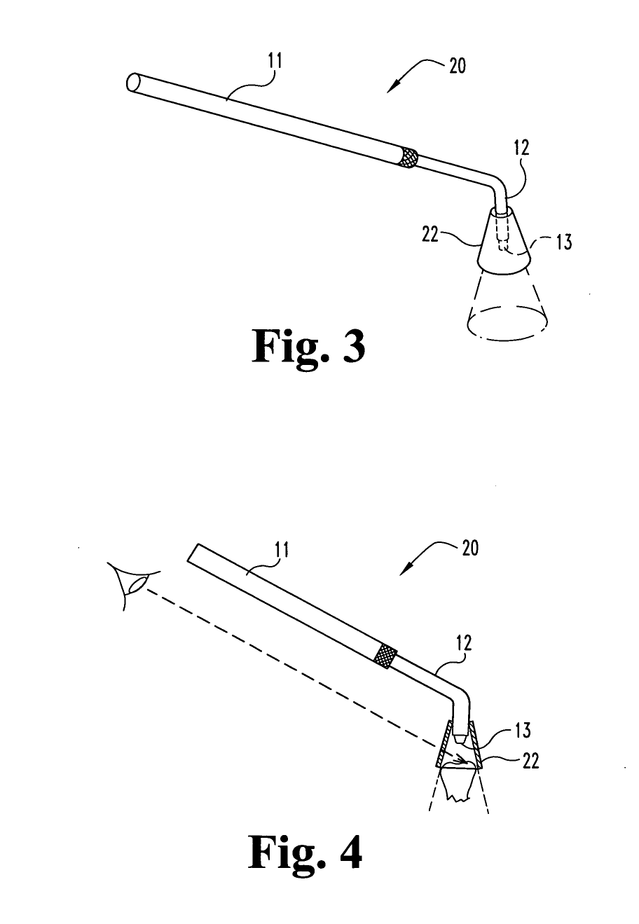

A second embodiment is illustrated in FIGS. 3 and 4. There dental implement 20 includes a filter structure 22 that has a conical shape and is attached to the tip of portion 12 adjacent to LED 13. Filter structure 22 has a narrow open end secured to tip portion 12 and a wide, open end facing the tooth surface. The dental implement 20 can be used in the same manner as dental implement 10 (see FIG. 1), and has the advantage that a wider tooth surface can be covered by filter structure 22 (as shown in FIG. 4), beyond the tooth covered by the first embodiment (as shown in FIG. 2).

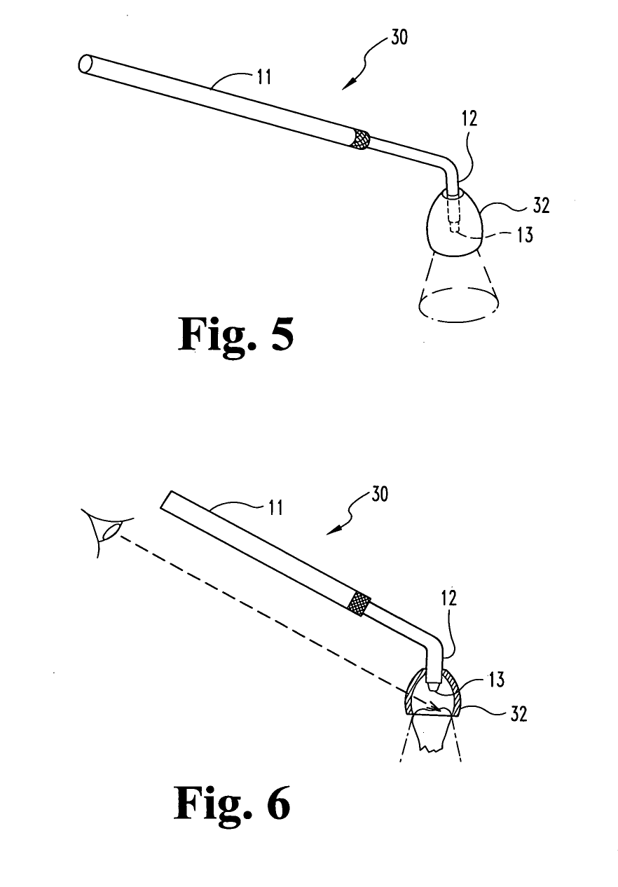

FIGS. 5 and 6 show a third embodiment. In FIG. 5, dental implement 30 includes a filter structure 32 having generally the shape of a partial sphere. Filter structure 32 is provided in the same manner as in the second embodiment, and has similar tooth-coverage capabilities as the second embodiment. In addition, filter structure 32 provides a magnifying function so as to yield a magnified view of the tooth surface, in addition to its filtering operation.

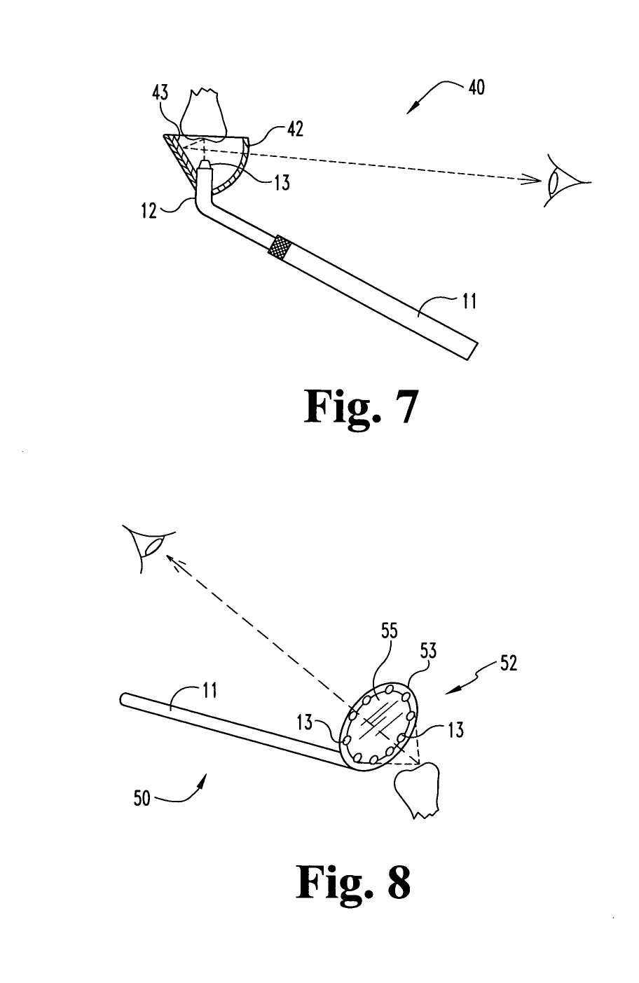

FIG. 7 shows a fourth embodiment, in which dental implement 40 has a filter structure 42 with a partly conical, partly hemispherical shape. In an inner surface of filter structure 42, a mirror 43 is provided that partially covers the inner surface of filter structure 42. In some forms of the fourth embodiment, a portion of the filter structure 42 that does not have a mirror 43 has magnifying properties. In some other forms, mirror 43 is coated with an anti-fog treatment (as are known to those skilled in the art) to reduce condensation or accumulation of liquid on the mirror surface. According to this fourth embodiment, dental implement 40 emits light from LED 13 to the tooth surface, and fluorescence is reflected in mirror 43 and observed through the filter structure 42.

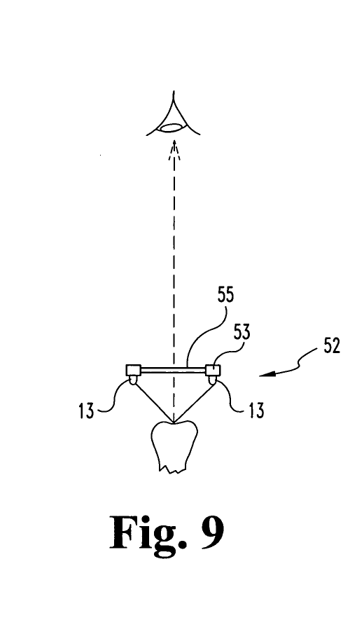

FIGS. 8 and 9 show a fifth embodiment. As shown in FIG. 8, dental implement 50 includes a handle portion 11 and LED/filter structure assembly 52 which is provided at an end of handle portion 11 at a predetermined angle. The LED/filter structure assembly 52 includes a ring 53, one or more LEDs 13, and filter 55. The LEDs 13 are provided on one side of ring 53 so as to illuminate the tooth surface, and the filter is provided inside the ring 53. To use dental implement 50, ring 53 is held near an open mouth or inserted into the mouth and positioned near a tooth surface, as shown in FIG. 9, so that LEDs 13 face the tooth surface. The light emitted by the LEDs 13 strikes the tooth, and fluorescence generated by the tooth is observed through filter 55. In dental implement 50, filter 55 may be formed to have magnifying properties in order to provide a magnified view of the tooth during use of the implement.

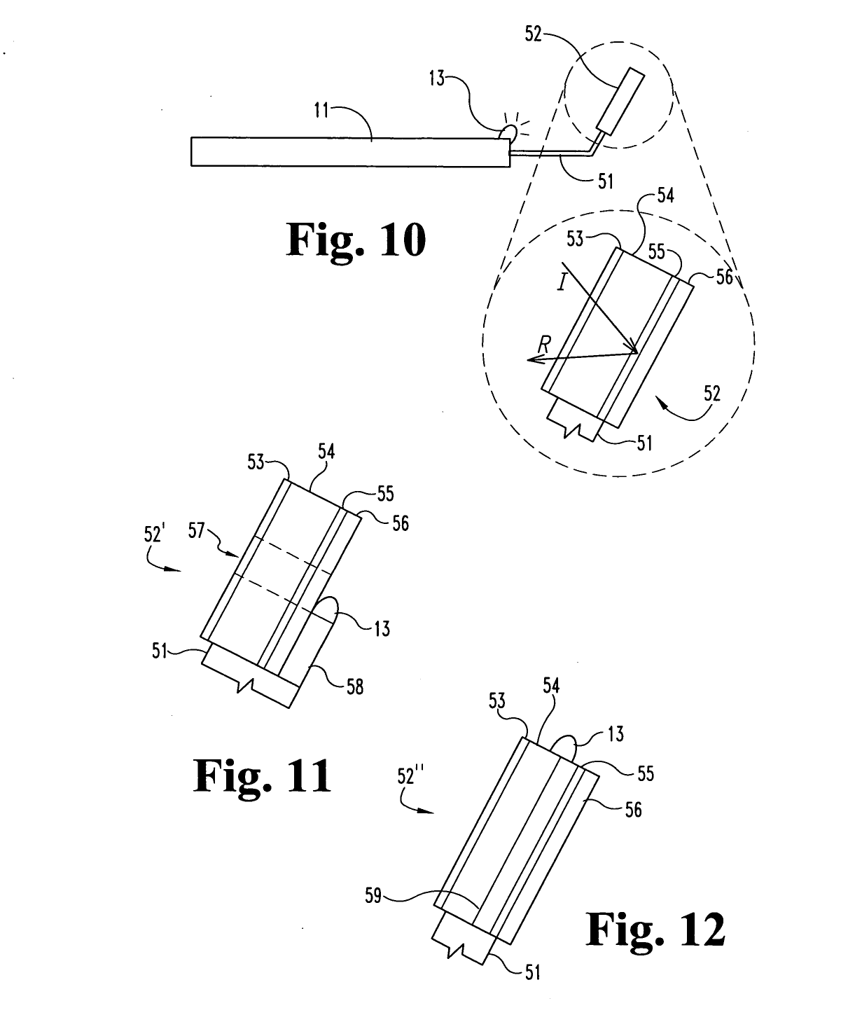

A sixth embodiment of the invention is shown in FIG. 10, with variations shown in FIGS. 11 and 12. In FIG. 10, light element 13 is mounted at the end of handle 11, along with a supporting structure 51 (such as a stiff wire) and optical element 52. Optical element 52 comprises layers 53-56, where layers 53 and 55 are anti-reflective layers, layer 54 is an optical filter, and layer 56 is a mirror coating layer. An exemplary ray of light incident on element 52 is shown in FIG. 10 and is labeled I. At least some of the incident light I in this embodiment is the fluorescent response of a bodily tissue to light emitted by element 13. Incident light I is filtered as it passes through filtering layer 54 so that desired wavelengths pass and others are absorbed. When incident ray I reaches mirror coating 56, it is reflected and becomes reflected ray R. Reflected ray R passes again through filter layer 54 to further attenuate undesired wavelengths, and passes back out of optical element 52, preferably to the eye of an observer. Anti-reflective layers 53 and 55 reduce attenuation of the viewed rays R due to internal reflection within optical element 52.

FIG. 11 illustrates an alternative optical element 52′, which comprises layers 53-56 that are analogous to those layers in FIG. 10. In optical element 52′, however, lighting element 13 lies behind reflective layer 56, and aperture 57 (through each of layers 53-56) allows lighting element 13 to pass through the remainder of optical element 52′ to reach the tissue subject to examination. The tissue autofluoresces (as is known in the art and mentioned above) and strikes optical element 52′ as incident light I, and reflects as reflected light R as was shown in relation to FIG. 10. The aperture 57 is a cylindrical bore, perpendicular to the surface of layer 53, through all layers of optical element 52′ in the center of the face. In alternative embodiments aperture 57 has other shapes and is placed in other locations and at other angles in optical element 52′ as would occur to one skilled in the art. Wires to pass energy to lighting element 13 pass through conduit 58 that lies across the back side of optical element 52′.

Another alternative embodiment includes optical element 52″ as shown in FIG. 12. In this embodiment, in addition to layers 53-56 (discussed in relation to FIG. 10), wire 59 extends around the perimeter of optical element 52″ to connect lighting element 13 with a power source through support member 51.

In FIGS. 11-12, energy to illuminate lighting element 13 passes through support member 51 to connect lighting element 13 with a power source such as a battery compartment in handle 11. In alternative embodiments, power is derived from an AC power supply, either with or without passing through a transformer or rectifier, as is necessary or preferred in those alternative implementations.

It will be observed by those of skill in the art that these mirror/filter combinations provide substantial advantages over gold mirrors and detached filters. As to the former, while a reflection in a gold mirror might have an augmented red component, it does not assist in observing the green component, making diagnosis more difficult. Further, the integrated mirror/filter of FIGS. 10-12 is easier to manage for the user than a standard mirror and a detached filter.

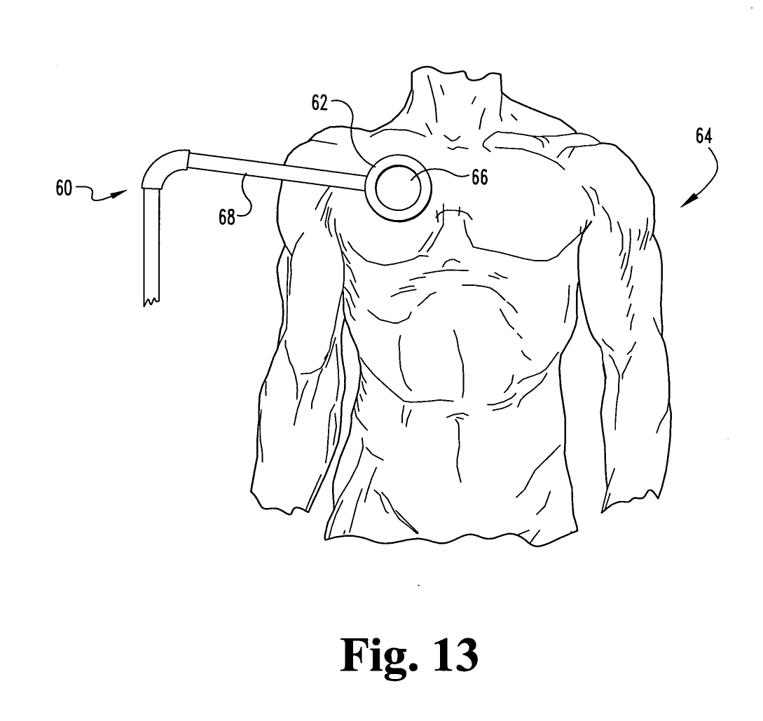

FIG. 13 illustrates an application of the present invention to dermatology. In this ninth embodiment, stand 60 supports loupe 62, which includes a ring of LEDs on one side for illuminating the skin of a subject 64. Filter/magnifier 66 fills the exterior ring 62 to provide a window through which a doctor or technician observes the response of skin lesions to the incident light from the LEDs. Support arm 68 is movable relative to support 60 so that the observer moves loupe 62 as necessary or desirable for inspection of the lesions.

In alternative forms of this embodiment, support 60 is permanently or removably attached to an examination table, wall, or light fixture. Support arm 68 is extendable in various forms of the embodiment. While the invention is illustrated in terms of examination of human subjects, and the invention is particularly advantageous when put to that purpose, it is also applied to other animal or vegetable tissues as would occur to one skilled in the relevant art.

It is noted in particular that some applications of the present invention will be in the field of dermatology. For example, cancer detection (both oral cancers and skin cancers) and acne/blackhead treatment can use the invention described and claimed herein.

It is also noted that the geometric descriptions of shields in this document apply primarily to the interior surfaces of the shields, though contouring and shaping of the exterior surfaces may be useful for aesthetics and applications of the present teachings regarding magnification. It is further noted that the traditional mathematical definition of “cylinder” is intended throughout this document; mainly, “a: the surface traced by a straight line moving parallel to a fixed straight line and intersecting a fixed planar closed curve, b: the space bounded by a cylinder and to parallel planes cutting all its elements.”

It will be understood by those skilled in the art that the above features can be combined with each other and/or with these features disclosed in the Inspection application. Those skilled in the art will be able to also implement light elements other than LEDs in various embodiments of the present invention.

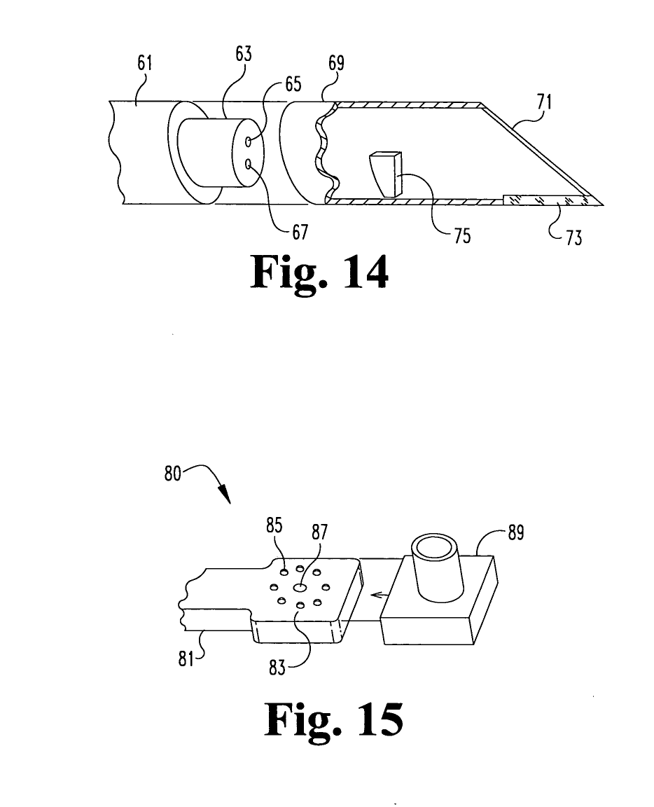

Turning to FIG. 14, a tenth embodiment of the present invention is shown. This embodiment relates to a shield, mirror, and filter arrangement that is well adapted for examination of dental structures and the capture of images in a controlled setting. In this embodiment, handheld portion 61 is generally cylindrical and ends in a narrower end portion 63. In the distal end surface of end portion 63 are a light source 65 and camera aperture 67. The former emits one or more wavelengths of light through source 65, while the latter (aperture 67) receives light for capture as an image. Aperture 67 may include a lens, one or more CCDs, and other components for image capture as would occur to one of ordinary skill in the art. Light source 65, likewise, may include one or more light-emitting diodes or the like, as would occur to one of ordinary skill in the art.

Light emitted by light source 65 travels through hood 69, which also has a generally cylindrical shape, but tapers in preferred embodiments to a narrower distal end. The emitted light from light source 65 is reflected off planar mirror 71 and through view port 73. The light induces autofluorescence by the dental tissue, returns through view port 73, reflects off mirror 71, and passes through optical filter 75 before passing through aperture 67 to be captured as an image.

It may be observed that in this preferred embodiment, the length of hood 69 and configuration of mirror 71, view port 73, and filter 75 allow the dental professional to position the device consistently in order to obtain consistently scaled and positioned images over time. For example, the professional may place view port 73 directly on a tooth surface, which placement ensures that a particular distance (from the subject at the bottom surface of the view port 73, to mirror 71, to aperture 67, which is a fixed distance) is maintained during the image capture process. Even more exact positioning can be obtained using the devices and techniques described in U.S. Pat. No. 6,597,934. In these example configurations, the body of hood 69 is constructed of an opaque material, such as plastic, and prevents substantially all ambient light from entering the interior and interfering with image capture at aperture 67. In certain image-capturing scenarios, the patient is able to close his or her lips around the hood 69 and block substantially all light from affecting the image capture in any way.

In other embodiments, hood 69 is disposable, and is discarded after use by a single patient. In other embodiments, a clear plastic cover is placed over a more permanent hood 69, and after use is discarded. In still other embodiments, hood 69 is more permanently affixed to hand piece 61, while in still other embodiments, hood 69 snaps into place at the end of hand piece 61, using one or more attachment mechanisms known to those in the mechanical arts.

In still other embodiments, optical filter 75 is omitted, and the image captured through aperture 67 is digitally analyzed to remove frequency components below a certain threshold (for example, 520 nm or 580 nm). Analysis of the image may proceed as described above.

FIG. 15 shows the distal end of a handheld light and camera device 80, including a neck portion 81, white LEDs 83, blue LEDs 85, camera aperture 87, and opaque distance-holding shield 89. A three-way switch 91 on neck portion 81 has three positions, wherein (1) all LEDs are off, (2) the blue LEDs are on and a yellow filter covers the inside of aperture 87, and (3) the white LEDs are on and a gray filter covers the inside of aperture 87. Thus, a professional is able to examine a patient using the white light and camera, then without even removing the implement from the patient's mouth, can acquire images of fluorescence by the patient's oral structure(s) to diagnose conditions of the tooth or other oral structure.

While the invention has been illustrated and described in detail in the drawings and foregoing description, the same is to be considered as illustrative and not restrictive in character, it being understood that only the preferred embodiments have been shown and described and that all changes and modifications that would occur to one skilled in the relevant art are desired to be protected. In addition, all patents, publications, prior and simultaneous applications, and other documents cited herein are hereby incorporated by reference in their entirety as if each had been individually incorporated by reference and fully set forth.

What is claimed is:

1. An implement for use in detecting one or more abnormal tissue conditions, comprising:
one or more light elements adapted to emit autofluorescence-inducing light onto tissue; and
a shield positioned adjacent to the one or more light elements, where the shield optically filters light that has been produced by the autofluorescence of the tissue.

2. The implement of claim 1, wherein the shield has a cylindrical shape.

3. The implement of claim 2, wherein the shield has the shape of a right cylinder.

4. The implement of claim 3, wherein the shield has the shape of a right circular cylinder.

5. The implement of claim 2, wherein the shield has the shape of a circular cylinder.

6. The implement of claim 1, wherein the shield has a conical shape.

7. The implement of claim 1, wherein the shield has a spherical shape.

8. The implement of claim 1, wherein at least a part of the shield magnifies the region of space within the shield as viewed from outside the shield.

9. The implement of claim 1, wherein a mirror is within the shield and positioned to reflect at least part of the light emitted by autofluorescence of the oral structure.

10. The implement of claim 1, wherein the shield filters visible light of predetermined frequencies, which visible light has been produced by autofluorescence of the oral structures.

11. The implement of claim 1, wherein the one or more light elements number at least four and are positioned in a ring configuration.

12. The implement of claim 11, wherein the one or more light elements number at least six.

13. The implement of claim 11, wherein the shield is in the plane defined by the ring.

14. The implement of claim 13, wherein the shield substantially fills the plane defined by the ring.

15. The implement of claim 13, wherein the shield comprises a magnifying lens.

16. The implement of claim 1, wherein the one or more light elements are light-emitting diodes.

17. The implement of claim 1, wherein the shield is treated to reduce fogging.

18. The implement of claim 1, wherein the light passing through the shield is observed directly by a person.

19. The implement of claim 1, wherein the light passing through the shield is captured by an image capture element.

20. The implement of claim 19, wherein the shield holds image capture element a predetermined distance from the subject

21. The implement of claim 1, wherein the tissue is dental tissue.

22. The implement of claim 1, wherein the tissue is dermal tissue.

23. The implement of claim 1, wherein the one or more light elements and shield are affixed to a handheld case.

24. The implement of claim 1, wherein the one or more light elements and shield are mechanically supported in a user-selectable position.

25. The implement of claim 1, wherein the tissue is human tissue.

26. The implement of claim 1, wherein the tissue is canine tissue.

27. The implement of claim 1, wherein the tissue is feline tissue.

28. The implement of claim 1, further comprising:
an additional one or more light elements adapted to emit white light onto the tissue; and
a switch to selectively enable either
the one or more light elements adapted to emit autofluorescence-inducing light; or
the additional one or more light elements adapted to emit white light.

29. An implement for use in detecting one or more abnormal tissue conditions, comprising:
one or more light elements adapted to emit fluorescence-inducing light onto bodily tissue; and
a shield positioned adjacent to the one or more light elements, where the shield attenuates the incidence of ambient light onto the tissue for ambient light wavelengths that induce autofluorescence of the tissue at one or more wavelengths of interest.

30. The implement of claim 29, wherein the emissions of the light elements have a peak intensity at a wavelength between about 380 nm and about 500 nm.

31. The implement of claim 30, wherein the emissions of the light elements have a peak intensity at a wavelength between about 390 nm and about 450 mn.

32. The implement of claim 29, wherein:
the one or more light elements are incorporated into a handheld unit; and
the shield is removably attached to the handheld unit.

33. The implement of claim 29, wherein the light passing through the shield is observed directly by a person.

34. The implement of claim 29, wherein the light passing through the shield is captured by an image capture element.

35. The implement of claim 34, wherein the shield holds the image capture element a predetermined distance from the tissue.

36. The implement of claim 29, wherein the shield does not substantially attenuate ambient light wavelengths that do not induce autofluorescence of the tissue at the one or more wavelengths of interest.

37. The implement of claim 29, wherein the tissue is dental tissue.

38. The implement of claim 29, wherein:
the tissue is dental tissue in an oral cavity; and
the shield allows enough light to escape the oral cavity to allow visual observation of the oral cavity while the one or more light elements are illuminating the oral cavity.

39. The implement of claim 29, wherein the tissue is dermal tissue.

40. The implement of claim 29, wherein the one or more light elements and shield are affixed to a handheld case.

41. The implement of claim 29, wherein the one or more light elements and shield are mechanically supported in a user-selectable position.

42. The implement of claim 29, wherein the tissue is human tissue.

43. The implement of claim 29, wherein the tissue is canine tissue.

44. The implement of claim 29, wherein the tissue is feline tissue.

45. The implement of claim 29, further comprising:
an additional one or more light elements adapted to emit white light onto the tissue; and
a switch to selectively enable either
the one or more light elements adapted to emit autofluorescence-inducing light; or
the additional one or more light elements adapted to emit white light.

46. A system comprising:
an illumination device used for the inspection of bodily tissue for the presence or absence of tissue abnormalities; and
a mirror having a light-filtering layer;
wherein the mirror substantially reflects the fluorescence light remitted from the tissue towards a detector; and
wherein the light-filtering layer substantially attenuates light of the wavelengths that induce the tissue fluorescence.

47. The system of claim 46, wherein the mirror magnifies the fluorescence light remitted from the tissue.

48. The system of claim 46, wherein the mirror has an anti-reflective coating.

49. The system of claim 48, wherein the mirror comprises:
a first anti-reflective coating layer,
a second anti-reflective coating layer,
an optical filter layer between the first and second anti-reflective coating layers, and
a mirror coating layer, where the second anti-reflective coating layer is between the optical filter layer and the mirror coating layer.

50. The system of claim 46, wherein the detector is a human eye.

51. The system of claim 46, wherein the detector is a camera.

52. The implement of claim 46, wherein the tissue is dental tissue.

53. The implement of claim 46, wherein the tissue is dermal tissue.

54. The implement of claim 46, wherein the shield is treated to reduce fogging.

55. The implement of claim 46, wherein
the illumination device comprises a plurality of light elements arranged as a ring; and
the mirror is situated within the ring.

56. The implement of claim 46, wherein the illumination device comprises at least one light element that emits light having a peak intensity between about 380 nm and about 500 nm.

57. The implement of claim 56, wherein the at least one light element emits light having a peak intensity between about 390 nm and about 450 nm.

58. The implement of claim 46, wherein the light-filtering layer substantially attenuates light having a wavelength less than about 520 nm.

59. The implement of claim 58, wherein the light-filtering layer substantially attenuates light having a wavelength less than about 580 nm.

60. The implement of claim 46, wherein the mirror is integrated into a single housing with the illumination device.

61. The implement of claim 46, wherein the mirror is detachable from the illumination device.
